(19) United States
(12) Patent Application Publication
(10) Pub. No.: US 2020/0030134 A1
(43) Pub. Date: Jan. 30, 2020

(54) FLANGE FOR OSTOMY BAG

(71) Applicant: WELLAND MEDICAL LIMITED, West Sussex (GB)

(72) Inventor: Jack HOPPER, West Sussex (GB)

(73) Assignee: WELLAND MEDICAL LIMITED, West Sussex (GB)

(21) Appl. No.: 16/487,728

(22) PCT Filed: Jan. 31, 2018
(86) PCT No.: PCT/EP2018/052368

§ 371 Date: Aug. 21, 2019

(30) Foreign Application Priority Data
Feb. 24, 2017 (GB) 1703046.1

(51) Int. Cl. A61F 5/443; A61F 5/448

(52) U.S. Cl. CPC A61F 5/443; A61F 5/448

(57) ABSTRACT

An adhesive flange (1) for an ostomy bag (4) comprises an adhesive first surface for facing the body of a user and a second surface for facing away from the body of a user said flange defining a general plane of reference a thickness thereof being defined in a direction perpendicular thereto. The flange has an inner edge which defines an opening (2) for receiving a stoma and an outer edge which defines a plurality of radial projections. A plurality of removable easy release liners (6) cover the adhesive until the ostomy bag is ready for use. The flange encourages an effective application of the ostomy bag by a user. The ostomy bag can be used to collect waste from a stoma of an ostomate.

The present invention relates to a flange for an ostomy bag as well as an ostomy bag comprising a pouch defined by two opposing surfaces of two walls, and a flange. The flange encourages an effective application of the ostomy bag by a user. The ostomy bag can be used to collect waste from a stoma of an ostomate.

### BACKGROUND OF THE INVENTION

Ostomy bags are medical devices that are worn by an individual and they can be used for the collection of waste from a surgically diverted bowel or urinary system of the individual. They are used to collect waste that is output from a stoma created in the ostomate's skin and connected to the intestine or urinary system.

Known ostomy bags comprise a pouch and, in some cases, the pouch is attached mechanically or with adhesive to a flange, commonly referred to as a mounting plate, wafer or a baseplate. The flange is commonly manufactured of hydrocolloid, which serves as an adhesive for attaching the ostomy bag to skin surrounding a stoma of an ostomate. Alternative known flanges are manufactured of an ultra-thin film having an adhesive applied to their body facing surface. In use, the flange is fixed to the skin of an individual and the ostomy bag allows the waste to drain from a stoma into the pouch while protecting the surrounding skin from contamination by the waste.

Ostomy bags should be air- and water-tight and they should allow the individual to lead an active normal lifestyle that can include all forms of sport and recreation.

People living with stomas sometimes incur leakage from their ostomy bags. Leakage most frequently occurs where the bag joins the body, under and around the flange. Leakage can occur for a variety of reasons including one or more of the following:
Poor Application
Adhesive Failure
Sore skin
Medication
Skin based products.

In addition, in some cases, the flange can become detached from the body when the ostomate moves. This can result in loss of, or decline in, adhesion of the ostomy bag. Loss of, or decline in, adhesion can have potentially difficult and embarrassing consequences for an ostomate due to the nature of the waste in the pouch and unpredictable output of effluent from the stoma.

The present invention seeks to provide a flange for an ostomy bag, which addresses one or more of the problems discussed above. In particular, the present invention seeks to provide a flange which encourages an effective application by a user.

### SUMMARY OF THE INVENTION

Remarkably, it has now been found that users adopt a variety of different techniques for applying an ostomy bag and a study has revealed that users who run their fingers around the outer perimeter of the flange in order to seal the flange to skin around a stoma achieve the least secure application of their ostomy bag, whereas those users whom adopted a radial technique of applying their ostomy bag, starting by applying pressure near the centre of the flange, running their fingers radially outwards towards the perimeter of the flange achieved the most secure application. In light of this, a new flange for an ostomy bag has now been produced with the aim of encouraging users to adopt the best possible technique for application of the flange to skin around a stoma.

Therefore, in accordance with a first aspect of the present invention, there is provided a flange for an ostomy bag having a shape for encouraging effective application thereof wherein the flange has an adhesive first surface for facing the body of a user and a second surface for facing away from the body of a user said flange defining a general plane of reference a thickness thereof being defined in a direction perpendicular thereto and having an inner edge which defines an opening for receiving a stoma and an outer edge which defines a plurality of radial projections.

Preferably, the outer edge defines only three, only four or only five radial projections. Most preferably, the outer edge defines four radial projections. Preferably, the radial projections are rounded and smoothed so that there are no sharp edges.

Preferably, the projections are the projections are equally spaced circumferentially.

Preferably, the outer edge defines a plurality of indents between each radial projection. Therefore, radial distance from the inner edge to the outer edge is greater at each projection and smaller at each indent.

Preferably, the outer edge defines a disk having indents and each indent is defined by a removed segment of the disk. Preferably, the disk is a circular disk.

Preferably each indent is curved and defined by a curved segment of the disk wherein the radial distance from the centre to the outer edge between each projection is reduced. In this regard, the radial distance to the outer edge is preferably tapered between each projection and at each indent to a radial distance from the centre of the disk of about 75% to about 95%, more preferably about 80% to about 90%, most preferably about 86% the maximum radial distance from the centre to the outer edge. In addition, each indent is defined by a circumferential distance removed from the disc between each projection. Preferably, the circumferential distance removed from the disc between each projection is about 2 to about 6, more preferably about 3 to about 5, more preferably about 4 times the difference between the radial distance from the centre to the outer edge at each indent and the radial distance from the centre to the outer edge at each projection.

Preferably, the outer edge at each indent is rounded and smoothed so that there are no sharp edges.

In a preferred embodiment, the outer edge defines the shape of a rounded cross. Advantageously, it has been found that this shape encourages a user to apply radial pressure on the flange during application to the skin and this has been found to result in an effective application of the flange.

In an embodiment, the flange is provided with a plurality of removable easy release liners which cover the adhesive until the ostomy bag is ready for use.

Preferably, only three, only four, only five or only six easy release liners cover the adhesive until the ostomy bag is ready for use. More preferably, only four or five easy release liners cover the adhesive until the ostomy bag is ready for use.

Preferably, each easy release liner comprises a tab which does not adhere to the adhesive. Preferably, each release liner projects radially outwardly. Advantageously each tab can be easily gripped to facilitate removal of the release liner from the adhesive.

Preferably, a first easy release liner covers the adhesive adjacent the inner edge of the flange until the ostomy bag is ready for use. Advantageously, removal of the first easy release liner enables a user to carry out preliminary application of the flange to their skin.

In addition, preferably, further easy release liners cover the adhesive adjacent the outer edge of the flange. Preferably, the adhesive on each radial projection is covered by a separate easy release liner.

In use, preferably the first release liner is removed and preliminary application of the flange to the skin is carried out. Thereafter, the further release liners are removed in series. In this regard, the release liners are removed radially by peeling them away and it has been found that this encourages a user to further apply the flange to the skin by exerting pressure on the flange radially after each release liner is removed.

Preferably, at least the first easy release liner is labelled with a label to indicate the order in which the release liners should be removed. More preferably, each easy release liner is labelled with a label to indicate the order in which the release liners should be removed. Preferably the label is either a number or a letter or both. More preferably, the label is a number. Preferably, the tab of the release liner is labelled with a label.

Preferably, the flange is transparent or translucent. This provides the advantage of being discreet and, in use, the skin where the flange is placed can be inspected.

In a first embodiment the flange is manufactured of hydrocolloid. Preferably, the flange has a thickness of about 0.5 mm to about 1.8 mm. Preferably, the adhesive is provided by the hydrocolloid.

In an alternative embodiment the flange is manufactured of an ultrathin Preferably, the flange has a thickness of about 5 μm to about 20 μm. Preferably, the adhesive is acrylic-based or silicone-based or PU-based. More preferably, the adhesive is acrylic-based.

Preferably, the easy release liner comprises a tab which does not adhere to the adhesive. Just before use, the easy release liner can be peeled away to expose the adhesive.

Advantageously, the tab is easy to grip and facilitates removal of the release liner.

In a second aspect, the invention provides an ostomy bag which comprises a pouch defined by two opposing surfaces of two walls and a flange as described above.

In another aspect, the invention provides a method for the production of the ostomy bag according to the invention, wherein the method comprises providing a flange as described above and attaching it to a pouch.

Advantageously, it has been found that the flange according to the invention assists with encouraging effective application of the flange to the skin. This provides increased security of application and improved comfort for a user.

### BRIEF DESCRIPTION OF THE DRAWINGS

The invention will now be described by way of example with reference to the accompanying drawings, in which:—

FIG. 1 shows flanges having different easy release liners. In FIG. 1a a known flange having a standard release liner is shown (A1) (Control); and in FIG. 1b a flange having a plurality of easy release liners is shown (A2).

FIG. 2 shows the shape of flanges according to the invention. In FIG. 2a, a flange according to the invention having a single easy release liner is shown (B1); and in FIG. 2b a flange according to the invention having a plurality of easy release liners is shown (B2).

FIG. 3 shows a graph of experimental results showing which design variant of flange achieved the fewest number of creases when applied. A1=existing known flange as shown in FIG. 1a; A2=flange having a plurality of easy release liners as shown in FIG. 1b; B1=flange of the invention having four radial projections and having a single easy release liner as shown in FIG. 2a; B2=a flange according to the invention having a plurality of easy release liners as shown in FIG. 2b.

FIG. 4 shows a graph of experimental results showing which design variants had the least unadhered surface area when applied. A1=existing known flange as shown in FIG. 1a; A2=flange having a plurality of easy release liners as shown in FIG. 1b; B1=flange of the invention having four radial projections and having a single easy release liner as shown in FIG. 2a; B2=a flange according to the invention having a plurality of easy release liners as shown in FIG. 2b.

FIG. 5 shows a graph of experimental results showing the number of participants in a study who adopted a radial application technique. A1=existing known flange as shown in FIG. 1a; A2=flange having a plurality of easy release liners as shown in FIG. 1b; B1=flange of the invention having four radial projections and having a single easy release liner as shown in FIG. 2a; B2=a flange according to the invention having a plurality of easy release liners as shown in FIG. 2b.

FIG. 6 shows a graph to show the effect of using a radial application technique on the number of creases present in the wafer after application. The line ending top right=application technique; and the line ending bottom right=number of creases.

### DETAILED DESCRIPTION OF THE INVENTION

It will be appreciated that aspects, embodiments and preferred features of the invention have been described herein in a way that allows the specification to be written in a clear and concise way. However, unless circumstances clearly dictate otherwise, aspects, embodiments and preferred features can be variously combined or separated in accordance with the invention. In a preferred embodiment, a device in accordance with the invention comprises all aspects of the invention.

Within the context of this specification, the word “about” means preferably plus or minus 20%, more preferably plus or minus 10%, even more preferably plus or minus 5%, most preferably plus or minus 2%.

Within the context of this specification, the word “comprises” means “includes, amongst other things” and should not be construed to mean “consists of only”.

Within the context of this specification, the word “substantially” means preferably at least 90%, more preferably 95%, even more preferably 98%, most preferably 99%.

Within the context of this specification, the word “plurality” means two or more; preferably three, four or five; most preferably four.

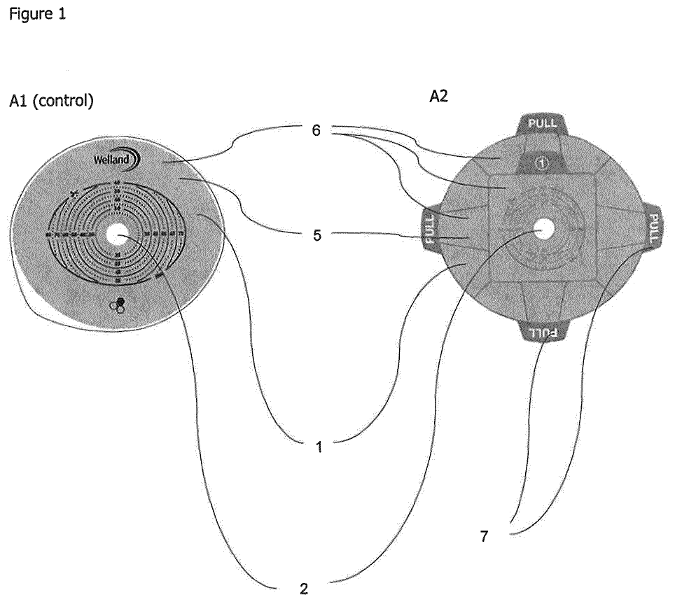

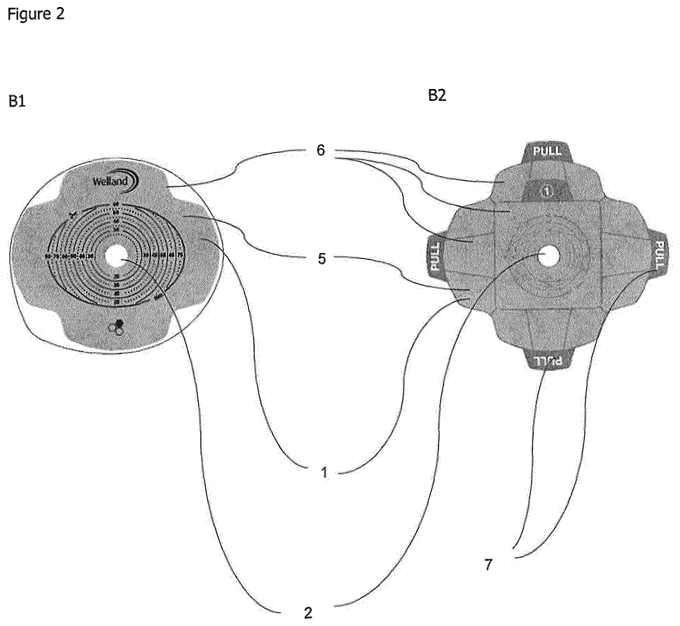

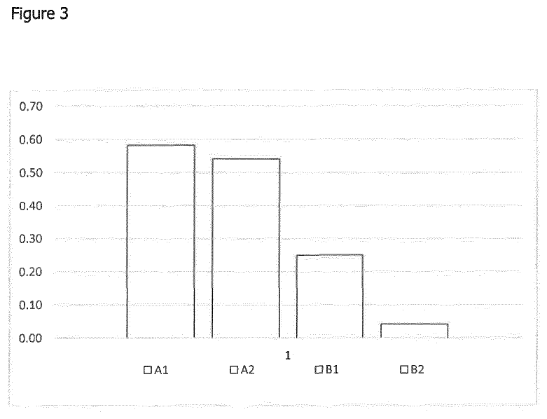

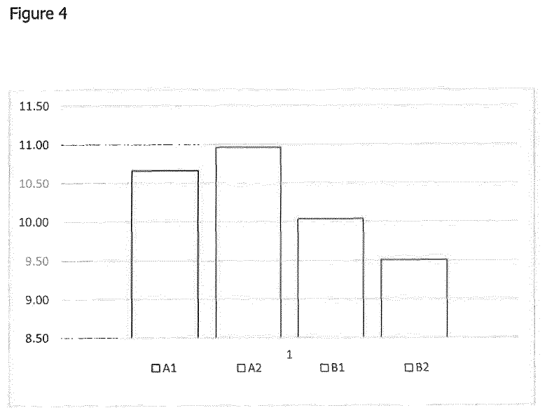

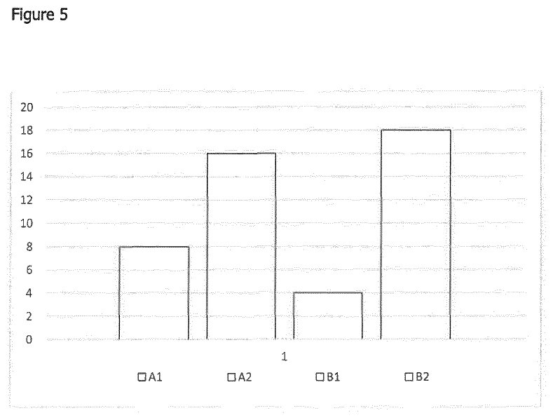

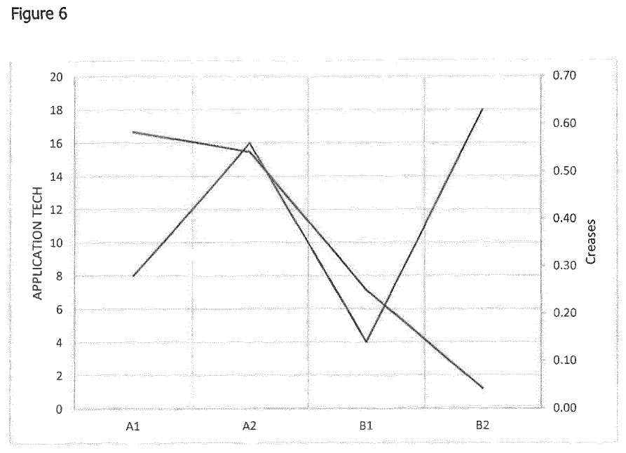

Referring to FIGS. 1 to 6, a flange (1) according to the invention for an ostomy bag defines an opening (2) for receiving a stoma. The flange (1) has a special shape as shown in FIG. 2.

In a first embodiment, the flange is manufactured of hydrocolloid and has a thickness of 0.5 mm to about 1.8 mm. The flange (1) adheres to the skin of an ostomate and is attached to a pouch (3) of an ostomy bag (4). In this regard, the surface of the hydrocolloid (5) adheres to the skin.

A series of easy release liners (6) cover the surface of the hydrocolloid (5). The easy release liners (6) can be peeled away prior to use to expose the surface of the hydrocolloid (5). This provides the advantage of protecting the surface of the hydrocolloid (5) until it is ready for use.

The easy release liner (6) comprises a tab (7) which does not adhere to the surface of the hydrocolloid (5). This provides the advantage that the tab (7) can be easily gripped and facilitates removal of the easy release liner (6) to expose the surface of the hydrocolloid (5).

To manufacture an ostomy bag according to the first embodiment a hydrocolloid flange (1) is provided and the flange (1) is attached to a pouch. An easy release liner (6) is applied to the surface of the hydrocolloid (5). The easy release liner has a tab (7) for easy removal.

In an alternative embodiment, the flange (1) is manufactured of an ultrathin film and has a thickness of about 5 μm to about 20 μm. The flange (1) adheres to the skin of an ostomate and is attached to a pouch (3) of an ostomy bag (4). In this regard, an acrylic based adhesive (5) is applied to the body facing surface of the flange.

A series of easy release liners (6) cover the adhesive (5). The easy release liners (6) can be peeled away prior to use to expose the adhesive (5). This provides the advantage of protecting the adhesive (5) until it is ready for use.

The easy release liner (6) comprises a tab (7) which does not adhere to the adhesive (5). This provides the advantage that the tab (7) can be easily gripped and facilitates removal of the easy release liner (6) to expose the adhesive (5).

To manufacture an ostomy bag, a flange (1) is provided. Acrylic adhesive (5) is applied to the flange (1) and attached to a pouch. Acrylic adhesive (5) is applied to the flange (1) and an easy release liner (6) is applied having a tab (7) for easy removal.

Experimental tests were carried out to assess a flange of the invention. In this regard, the objectives of the experimental tests were to assess the following:

1) If changes to the shape of the adhesive flange encourage the user to adopt a radial application technique.
2) If changes to the shape of the adhesive flange enable the user to achieve a better quality of application.
3) If changes to the structure of the release liner encourage the user to adopt a radial application technique.
4) If changes to the structure of the release liner enable the user to achieve a better quality of application.
5) Whether changes to the structure of the release liner or changes in the design of the flange shape have the biggest impact on application quality.
6) Whether changes to the structure of the release liner or changes in the design of the flange shape have the biggest impact on modifying the user's behaviour to adopt a radial application technique.

The following materials were used.

| Materials | Quantity |
|---|---|
| Known Ostomy bags having known | 60 (Clear Pouches) |
| standard hydrocolloid flange and known | |
| release liner | |
| Ostomy bags having a flange with four | 60 (Clear Pouches) |
| radial projections | |
| Ostomy bags having a flange with four | 30 (Clear Pouches) |
| radial projections and series of easy | |
| release liners | |
| Known Ostomy bags having known | 30 (Clear Pouches) |
| standard hydrocolloid flange, but having | |
| a series of release liners | |
| Synthetic Hernia | 4 |
| Adhesive Remove Spray | 1 |
| Dry Wipe Marker | 1 |
| Camera | 1 |
| Camera Tripod | 1 |
| Elasticated Belt | 4 |

The following test conditions were used.

| Category | Variables |
|---|---|
| Flange Shape | a standard known flange (A1); and |
| | a flange having four radial projections (B1) |
| Release Liners | a standard known release liner (A1) |
| | (Control); and a series of separate |
| | release liners having tabs (A2) |

Two type of release liner were used: a standard known release liner (A1) (Control) and a series of separate release liners having tabs (A2).

Two varieties of flange shapes were used: a standard known shape flange (A1) and a flange having four radial projections (B1).

Finally, a variety of flange (B2) was used wherein a series of release liners each having a tab (as in A2) and the shape of the flange had four radial projections (as in B1).

Due to the varying body types amongst volunteers a synthetic hernia was used to draw a fair comparison between the performance of the tested release liners and flange shapes.

Volunteers were prevented from applying flanges attached to standard opaque pouches as this would have made it impossible to film and observe the volunteers application technique fully. Instead, volunteers were asked to apply flanges which were attached to clear colourless bags so that information could be captured successfully.

Volunteers were instructed only to apply the flange to the synthetic hernia so as to minimise the risk of leading the volunteers to exhibit a specific behaviour.

The protocol for the experimental tests was as follows:
1) Volunteers were asked to strap the synthetic hernia around their waist.
2) Each volunteer was handed one of the test flanges and asked to apply the flange to the synthetic hernia.
3) Once the volunteer had applied the flange to the synthetic hernia, the volunteer was asked to remove the synthetic hernia with the flange still attached.
4) The number of folds in the flange present around the perimeter were counted and logged.
5) The areas where the flange had not fully adhered to the synthetic surface was then marked and photographed for later analysis.
6) The volunteers were asked to repeat this process for each of the three remaining test flanges.
7) After the volunteers had tested all 4 samples the synthetic hernia was cleaned ready to be used for testing again.
8) This process was repeated for all volunteers.

Data analysis was carried out as follows:

| Output | Method of Interpretation |
|---|---|
| Application | Volunteers were filmed when applying their flanges. |
| Technique | This footage was analysed after the testing had been |
| | completed. |
| Number of | After volunteers had applied all of the test flanges the |
| Folds | number of folds present in the surface of each flange was |
| | counted. |
| Adherence | After each volunteer had removed the synthetic |
| | hernia with the flange still attached, the flange application |
| | area was observed and any areas of the flange which had |
| | not fully adhered were highlighted with a marker. The |
| | area was then photographed and stored. Once all testing |
| | had been completed all of the stored images were |
| | imported into an photo processing software package |
| | where all of the areas highlighted were coloured black. |
| | These images were then imported into image processing |
| | software and the percentage of the surface area of the |
| | flange successfully adhered to the synthetic surface was |
| | calculated. |

The results of the experimental tests identified that both the modified series of release liners and flange shape reduced the number of creases that occurred during the application process. The results of the experimental tests also highlighted that changing the flange shape to incorporate reliefs reduced the risk of creases being formed by 46.8% over modifying the release liner alone which achieved only a 6.9% reduction in the number of creases.

However when the modified release liner and the shape of the flange were combined, remarkably this resulted in a reduced risk of creases forming by a further 46.3% over only changing the shape of the flange, resulting in a 93.1% reduction in the formation of creases overall.

With regard to overall application, the shape of flange B2 showed a 1.15% improvement over A1. This demonstrated an improvement in the overall surface area of the flange in contact with the simulated hernia.

Importantly, the experimental tests identified that participants who adopted a radial application technique, applying the centre of the flange first were less likely to incur creases. The results also showed that the flange shape B2 was the flange variant most likely to encourage participants to adopt a radial application technique.

Furthermore, as is suggested from the results shown in FIG. (3), FIG. (6) also suggests that changing the shape of the flange has a greater impact on reducing the number of creases present in the surface of the flange over the multi stage release liner. However a combination of both shape change and multistage release does produce improved results.

In conclusion, the experimental tests highlighted that changes made to the release liner were more effective in encouraging participants to adopt a radial application technique than changes made to the shape of the flange. Changing the shape of the flange was more effective in reducing the likelihood of creases forming during the application process. When the changes made to the shape of the flange were coupled with changes made to the shape of the release liner the likelihood of creases forming were further decreased.

With regards to the overall surface area successfully applied to the artificial hernia. The shape of the flange was more successful as a standalone design modification over the multistage release liner, however when these features were combined they achieved an even more successful outcome.

Modifying the release liner and the flange shape had a positive impact of the percentage of the surface area successfully applied to the flange.

The above described embodiments have been given by way of example only, and the skilled reader will naturally appreciate that many variations could be made thereto without departing from the scope of the claims.

1-30. (canceled)

31. A flange for an ostomy bag having a shape for encouraging effective application thereof wherein the flange has an adhesive first surface for facing the body of a user and a second surface for facing away from the body of a user said flange defining a general plane of reference a thickness thereof being defined in a direction perpendicular thereto and having an inner edge which defines an opening for receiving a stoma and an outer edge which defines a plurality of radial projections.

32. A flange according to claim 31, wherein the outer edge defines only three, only four or only five radial projections; preferably four radial projections.

33. A flange according to claim 31, wherein the radial projections are (1) rounded and smoothed so that there are no sharp edges; and/or (2) equally spaced circumferentially.

34. A flange according to claim 31, wherein the outer edge defines (1) a plurality of indents between each radial projection; and/or (2) a disk having indents and each indent is defined by a removed segment of the disk.

35. A flange according to claim 34, wherein each indent is curved and defined by a curved segment of the disk wherein the radial distance from the centre of the flange to the outer edge between each projection is reduced; optionally wherein the radial distance from the centre of the flange to the outer edge is preferably tapered between each projection and at each indent to a radial distance from the centre of the flange of about 75% to about 95% the maximum radial distance from the centre to the outer edge; and/or optionally wherein each indent is defined by a circumferential distance removed from the disc between each projection.

36. A flange according to claim 35, wherein the circumferential distance removed from the disc between each projection is about 2 to about 6 times the difference between the radial distance from the centre of the flange to the outer edge at each indent and the radial distance from the centre to the outer edge at each projection.

37. A flange according to claim 34, wherein the outer edge at each indent is rounded and smoothed so that there are no sharp edges.

38. A flange according to claim 31, wherein the outer edge defines the shape of a rounded cross.

39. A flange according to claim 31, wherein the flange is provided with a plurality of removable easy release liners which cover the adhesive until the ostomy bag is ready for use.

40. A flange according to claim 39, wherein only three, only four, only five or only six easy release liners cover the adhesive until the ostomy bag is ready for use.

41. A flange according to claim 39, wherein each easy release liner comprises a tab which does not adhere to the adhesive.

42. A flange according to claim 41, wherein the tab of each release liner projects radially outwardly.

43. A flange according claim 39, wherein a first easy release liner covers the adhesive adjacent the inner edge of the flange until the ostomy bag is ready for use.

44. A flange according to claim 43, wherein one or more further easy release liners cover the adhesive adjacent the outer edge of the flange.

45. A flange according to claim 39, wherein the adhesive on each radial projection is covered by a separate easy release liner.

46. A flange according to claim 44, wherein in use, the first release liner is removed and preliminary application of the flange to the skin is carried out and thereafter, the further release liners are removed in series.

47. A flange according to claim 39, wherein at least the first easy release liner is labelled with a label to indicate the order in which the release liners should be removed.

48. A flange according to claim 31, wherein the flange is:
(i) transparent or translucent,
(ii) manufactured of hydrocolloid; optionally
wherein the flange has a thickness of about 0.5 mm to about 1.8 mm, or
(iii) manufactured of an ultrathin film; optionally
wherein the flange has a thickness of about 5 μm to about 20 μm.

49. An ostomy bag which comprises a pouch defined by two opposing surfaces of two walls and a flange according to claim 31.

50. A method for the production of an ostomy bag, wherein the method comprises providing a flange according to claim 31 and attaching it to a pouch defined by two opposing surfaces of two walls.
